(19) United States
(12) Patent Application Publication
(10) Pub. No.: US 2007/0243196 A1
(43) Pub. Date: Oct. 18, 2007

(54) Mage-3 and NY-ESO-1 Based Polyvalent Vaccine for Cancer Immunotherapy

(75) Inventors: Claudine Elvire Marie Bruck, King of Prussia, PA (US); Vincent Brichard, Rixensart (BE); Remi M. Palmantier, Rixensart (BE); Melinda Meaders, Rixensart (BE)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: GLAXOSMITHKLINE BIOLOGICALS S.A., Rixensart (BE)

(21) Appl. No.: 11/568,566

(22) PCT Filed: May 2, 2005
(86) PCT No.: PCT/EP05/04956

§ 371 Date: Nov. 1, 2006

(30) Foreign Application Priority Data
May 4, 2004 (GB) 0409940.4

(51) Int. Cl. A61K 39/00; A61K 39/39

(52) U.S. Cl. 424/184.1; 424/185.1

(57) ABSTRACT

The present invention provides novel vaccine formulations for the treatment of cancer antigens. The vaccine comprises a modified MAGE-3 antigen, a NY-ESO-1 antigen, and an adjuvant comprising a saponin and a immunostimulatory oligonucleotide.

The present invention relates to a novel vaccine formulation comprising (a) an antigen component comprising a combination of a modified MAGE-3 antigen and an NY-ESO-1 antigen, or derivatives thereof, and (b) an adjuvant.

Despite enormous investments of financial and human resources, cancer remains one of the major causes of death.

Immunotherapy of cancer has been described in the art for a number of years, including those comprising active vaccination of patients with tumour associated antigens with the aim to raise an immune response in these individuals which recognises and destroys the cancer cells. Many cancer antigens have been described for this purpose, including the MAGE family antigens and NY-ESO-1 antigens.

There remains, despite the length of time that these therapies have been investigated, a real need for improved strategies for enhancing the immune response against the antigen. Such strategies including the combination of the tumour antigen with powerful vaccine adjuvants.

Cancer/testis (CT) antigens are immunogenic proteins expressed predominantly in a variety of cancers but not in normal tissues except the gametogenic tissue (testis) (Kirkin, A et al. Cancer Investigation, 2002, 20(2), 222-236). MAGE-3 and NY-ESO-1 are known as the prototype CT antigens. The family of CT antigens also includes members of the NY-ESO-1, PRAME, GAGE family, PAGE family, BAGE, XAGE family, LAGE, members of the SSX family (amongst which SSX-1, -2 also known as HOM-MEL-40, -4, -5), SCP-1 (also known as HOM-TES-14), SART-1 and SART-3, HOM-TES-85, sperm-protein Sp17, CTp11, TSP50, CT9/BRDT, TRAG-3 (Taxol Resistance Associated Gene-3), OY-MS-4MAGE (see Table 1 in Kirkin A. et al. Cancer Investigation, 2002, 20(2), 222-236).

### SUMMARY OF THE INVENTION

The present invention relates to novel vaccine formulations comprising:

(a) an antigen component comprising combination of a modified MAGE-3 antigen and an NY-ESO-1 antigen or derivative thereof, and

(b) an immunostimulatory adjuvant comprising one or more of: alum salt; cholesterol; oil-in-water emulsion (O/W emulsion); oil-in-water emulsion low dose; an immunostimulatory oligonucleotide; tocopherol; liposome; a saponin; and a lipopolysaccharide.

Methods of treatment of individuals by administration of the vaccines of the present invention are also provided, and in specific embodiments the vaccines are used in the treatment of Melanoma, non-small cell lung carcinoma (NSCLC), or bladder cancer.

In one aspect of the present invention, there is provided a vaccine composition comprising (a) an antigen component comprising a fusion protein of MAGE 3 and a truncated Protein D carrier protein (MAGE3-ProteinD ⅓) of SEQ ID NO:1 (as described both herein and as described in WO 99/40188), an NY-ESO-1 protein antigen, and (b) an adjuvant composition comprising liposome structures containing cholesterol and QS21, in combination with an immunostimulatory oligonucleotide which contains at least one unmethylated oligonucleotide.

The vaccines of the present invention may improve the antitumour effect of the cancer vaccines in comparison with a vaccine containing only one of the antigens expressed by a tumour cell. This improved vaccine would not necessarily enable greater patient coverage (ie., allow more cancers to be targeted with one vaccine), but also allow a better immune response to be generated against the targeted tumour. In addition the vaccines of the present invention may reduce the chance of tumour evasion or escape, even if expression of one of the antigens is reduced after vaccination.

### DETAILED DESCRIPTION OF THE INVENTION

The invention relates to the specific combination of the following components:
(i) modified MAGE protein (MAGE3-ProteinD ⅓), as shown in SEQ ID NO:1
(ii) an “immunogenic region” of NY-ESO1 gene product, for example: the NY-ESO1 protein; a protein, polypeptide or peptide consisting of or comprising the C terminal portion of the protein containing the Class I and/or Class II epitopes of NY-ESO1; overlapping long peptides comprising this region; and/or, specific CD8 peptides.
(iii) an immunostimulatory adjuvant comprising one or more of: alum salt; cholesterol; oil-in-water emulsion (O/W emulsion); oil-in-water emulsion low dose; an immunostimulatory oligonucleotide, for example CpG; tocopherol; liposome; a saponin, for example QS21; and a lipopolysaccharide, for example MPL. Examples of adjuvants suitable for use in the present invention include those comprising or consisting of the following components:
   a. CpG; O/W emulsion/3D-MPL/QS21 (high dose);
   b. CpG/O/W emulsion low dose/3D-MPL/QS21;
   c. CpG/Liposome/QS21; and
   d. CpG/3D-MPL/QS21/liposomes.
   e. QS21-containing ISCOMS
   f. ISCOMS comprising QS21 and QS7

In one embodiment, the adjuvant is: CpG/MPL/QS21/liposomes

Components (i) and (ii) may be co-formulated with component (iii) for concomitant administration, or may each be separately formulated with component (iii) for concomitant or sequential administration.

In one embodiment of the present invention, component (i) is formulated with an adjuvant component (iii) which comprises CpG/MPL/QS21/liposomes and component (ii) is formulated with an adjuvant component (iii) which comprises ISCOMS, for example QS21-containing ISCOMS or ISCOMS comprising QS21 and QS7. Thus, component (i) and (ii) are provided for concomitant or sequential administration.

Components (i) and (ii) may be expressed as separate components, or may be expressed as a fusion protein. A DNA/viral vector vaccine is also envisaged, in which the vaccine comprises nucleic acid encoding components (i) and (ii), and component (iii) is a suitable adjuvant for a DNA vaccine.

The vaccine compositions comprise a MAGE-3 derivative antigen. In one embodiment of the present invention, the derivative is a fusion protein comprising a MAGE-3 antigen linked to a heterologous partner. The proteins may be chemically conjugated, or may be expressed as recombinant fusion proteins thus allowing increased levels to be produced in an expression system as compared to non-fused protein. Thus the fusion partner may assist in providing T helper epitopes (immunological fusion partner), for example T helper epitopes recognised by humans, or assist in expressing the protein (expression enhancer) at higher yields than the native recombinant protein. In one embodiment, the fusion partner will be both an immunological fusion partner and expression enhancing partner.

In one form of the invention, the MAGE-3 immunological fusion partner is derived from protein D, a surface protein of the gram-negative bacterium, Haemophilus influenza B (WO91/18926). In one embodiment, the protein D derivative comprises approximately the first ⅓ of the protein, in particular approximately the first N-terminal 100-110 amino acids. In one embodiment the protein D derivative is lipidated. In one embodiment the first 109 residues of the Lipoprotein D fusion partner is included on the N-terminus to provide the vaccine candidate antigen with additional exogenous T-cell epitopes and increase expression level in E-coli (thus acting also as an expression enhancer). The lipid tail ensures optimal presentation of the antigen to antigen presenting cells.

Other MAGE-3 fusion partners include the non-structural protein from influenzae virus, NS1 (hemagglutinin). Typically the N terminal 81 amino acids are utilised, although different fragments may be used provided they include T-helper epitopes.

In another embodiment the MAGE-3 immunological fusion partner is the protein known as LYTA. In one embodiment the C terminal portion of the molecule is used. Lyta is derived from Streptococcus pneumoniae which synthesize an N-acetyl-L-alanine amidase, amidase LYTA, (coded by the lytA gene {Gene, 43 (1986) page 265-272} an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at its amino terminus has been described {Biotechnology: 10, (1992) page 795-798}. As used herein, one embodiment of the invention utilises the repeat portion of the Lyta molecule found in the C terminal end starting at residue 178. One form which may be used incorporates residues 188-305.

In one embodiment of the present invention the modified MAGE-3 composition comprises an antigen as disclosed in WO 99/40188, or immunogenic fragment such as a peptide having retained the capability of eliciting an immune response which recognises the MAGE protein. A specific antigen for the present vaccines is the MAGE-3 polypeptide having the amino acid sequence set forth in Gaugler B. et al., J. Exp. Med., 1994, 179, 921 (MAGE-3), or in SEQ ID NO:1 (protein D⅓-MAGE-3) (both herein and also in WO 99/40188). Said immunogenic composition can be prepared according to the method disclosed in WO 99/40188 or by any routine technique known to the skilled in the art.

NY-ESO-1 is a tumour associated antigen described in WO 98/14464, the contents of which are incorporated in full into this disclosure. NY-ESO-1 is also described in Chen Y T et al., Proc Natl Acad Sci USA 1997, 94: 1914-18; Scanlan et al., 2004, Cancer Immunity, 4, 1. The protein and polynucleotide sequence for NY-ESO-1 is provided in Genbank ACCESSION No. U87459, Version U87459.1 (SEQ ID Nos 2 and 3).

The vaccine adjuvant that forms part of the present invention comprises an immunostimulatory adjuvant comprising one or more of: alum salt; cholesterol; oil-in-water emulsion (O/W emulsion); oil-in-water emulsion low dose; an immunostimulatory oligonucleotide; tocopherol; liposome; a saponin; and a lipopolysaccharide. In one embodiment, the adjuvant comprises an immunostimulatory oligonucleotide, a saponin, and optionally a derivative of Lipopolysaccharide (LPS). Optionally, the vaccine of the present invention may further comprise a carrier.

Immunostimulatory oligonucleotides containing unmethylated CpG dinucleotides (“CpG”) and are known in the art as being adjuvants when administered by both systemic and mucosal routes (WO 96/02555, EP 468520, Davis et al, J. Immunol, 1998, 160(2):870-876; McCluskie and Davis, J. Immunol., 1998, 161(9):4463-6). CpG is an abbreviation for cytosine-guanosine dinucleotide motifs present in DNA. Historically, it was observed that the DNA fraction of BCG could exert an anti-tumour effect. In further studies, synthetic oligonucleotides derived from BCG gene sequences were shown to be capable of inducing immunostimulatory effects (both in vitro and in vivo). The authors of these studies concluded that certain palindromic sequences, including a central CG motif, carried this activity. The central role of the CG motif in immunostimulation was later elucidated in a publication by Krieg, Nature 374, p 546 1995. Detailed analysis has shown that the CG motif has to be in a certain sequence context, and that such sequences are common in bacterial DNA but are rare in vertebrate DNA. The immunostimulatory sequence is often: Purine, Purine, C, G, pyrimidine, pyrimidine; wherein the dinucleotide CG motif is not methylated, but other unmethylated CpG sequences are known to be immunostimulatory and may be used in the present invention.

In certain combinations of the six nucleotides a palindromic sequence is present. Several of these motifs, either as repeats of one motif or a combination of different motifs, can be present in the same oligonucleotide. The presence of one or more of these immunostimulatory sequence containing oligonucleotides can activate various immune subsets, including natural killer cells (which produce interferon γ and have cytolytic activity) and macrophages (Wooldrige et al Vol 89 (no. 8), 1977). CpG when formulated into vaccines, is generally administered in free solution together with free antigen (WO 96/02555; McCluskie and Davis, supra) or covalently conjugated to an antigen (PCT Publication No. WO 98/16247), or formulated with a carrier such as aluminium hydroxide ((Hepatitis surface antigen) Davis et al. supra; Brazolot-Millan et al., Proc. Natl. Acad. Sci., USA, 1998, 95(26), 15553-8).

In one aspect of the present invention the oligonucleotides for use in the vaccines of the present invention may contain at least one unmethylated CpG motifs separated by at least three, or at least six or more nucleotides. The oligonucleotides of the present invention are typically deoxynucleotides. In one embodiment the internucleotide in the oligonucleotide is phosphorodithioate. In another embodiment, the internucleotide is a phosphorothioate bond. However, phosphodiester and other internucleotide bonds are within the scope of the invention including oligonucleotides with mixed internucleotide linkages. Methods for producing phosphorothioate oligonucleotides or phosphorodithioate are described in U.S. Pat. No. 5,666,153, U.S. Pat. No. 5,278,302 and WO95/26204.

Examples of oligonucleotides which may be used have the following sequences. The sequences may contain phosphorothioate modified internucleotide linkages.

OLIGO 1 (SEQ ID NO:4):
TCC ATG ACG TTC CTG ACG TT
(CpG 1826)
OLIGO 2 (SEQ ID NO:5):
TCT CCC AGC GTG CGC CAT
(CpG 1758)
OLIGO 3 (SEQ ID NO:6):
ACC GAT GAC GTC GCC GGT GAC GGC ACC ACG
OLIGO 4 (SEQ ID NO:7):
TCG TCG TTT TGT CGT TTT GTC GTT
(CpG 2006)
OLIGO 5 (SEQ ID NO:8):
TCC ATG ACG TTC CTG ATG CT
(CpG 1668)

Alternative CpG oligonucleotides may comprise the sequences above in that they have inconsequential deletions or additions thereto.

The CpG oligonucleotides utilised in the present invention may be synthesized by any method known in the art (e.g. EP 468520). Conveniently, such oligonucleotides may be synthesized utilising an automated synthesizer. They are typically between 10-50 bases in length.

The oligonucleotides utilised in the present invention are typically deoxynucleotides. In one embodiment the internucleotide bond in the oligonucleotide is phosphorodithioate, or more for example phosphorothioate bond, although phosphodiesters are within the scope of the present invention. Oligonucleotide comprising different internucleotide linkages are contemplated, e.g. mixed phosphorothioate phophodiesters. Other internucleotide bonds which stabilise the oligonucleotide may be used.

The saponins which may be used in the vaccine combinations of the present invention include those derived from the bark of Quillaja Saponaria Molina, termed Quil A, and fractions thereof, described in U.S. Pat. No. 5,057,540 and “Saponins as vaccine adjuvants”, Kensil, C. R., Crit Rev Ther Drug Carrier Syst, 1996, 12 (1-2):1-55; and EP 0 362 279 B1. Examples of suitable fractions of Quil A are QS21, QS7, and QS17. The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1. Also described in these references is the use of QS7 (a non-haemolytic fraction of Quil-A) which acts as a potent adjuvant for systemic vaccines. Use of QS21 is further described in Kensil et al. (1991, J. Immunology vol 146, 431-437). Combinations of QS21 and polysorbate or cyclodextrin are also known (WO 99/10008).

Particulate structures, termed Immune Stimulating Complexes (ISCOMS), comprising fractions of Quil A are haemolytic and have been used in the manufacture of vaccines (Morein, B., EP 0 109 942 B1). These structures have been reported to have adjuvant activity (EP 0 109 942 B1; WO 96/11711). Combinations of QS21 and polysorbate or cyclodextrin are also known (WO 99/10008). Particulate adjuvant systems comprising fractions of QuilA, such as QS21 and QS7 are described in WO 96/33739 and WO 96/11711.

In one embodiment of the present invention, the adjuvant component comprises a QS21-containing ISCOM. In a further embodiment, the adjuvant component comprises ISCOMS comprising QS21 and QS7.

The adjuvant combinations of the present invention may further comprise a carrier, the carrier may be simply admixed with the adjuvants or alternatively the adjuvants may be associated with a particulate carrier entity to enhance the adjuvanticity of the combination. Systemic vaccines may, for example, comprise a carrier molecule. Exemplary carriers include mineral salts (for example, but not restricted to, aluminium or calcium salts), liposomes, ISCOMs, emulsions (oil in water, water in oil, water in oil in water), polymers (such as, but not restricted to polylactic, polyglycolic, polyphosphazine, polyaminoacid, alginate, chitosan) or microparticles. The vaccines of the present invention further comprise an antigen which may be associated with the CpG-carrier complex, or may not be associated with the CpG-carrier complex. In this case, the antigen may be free suspension or associated with a separate carrier.

The saponins forming part of the present invention may be separate in the form of micelles, or may be in the form of large ordered structures such as ISCOMs (EP 0 109 942 B1) or liposomes when formulated with cholesterol and lipid (“DQ” described in WO 96/33739), or in the form of an oil in water emulsion (WO 95/17210). The saponins may be associated with a metallic salt, such as aluminium hydroxide or aluminium phosphate (WO 98/15287). Alternatively the saponin may be associated with a particulate carrier such as chitosan. The saponin may also be in a dry state such as a powder. The final formulations in the form as they are administered to the mucosal surface of the vaccinee may be haemolytic in nature. The saponin may or may not be associated physically with the antigen either through direct linkage or by co-interaction with the same particulate carrier molecule (GB9822712.7; WO 98/16247).

The CpG and saponin which may be used in the adjuvants or vaccines of the present invention may themselves be separate or associated. For example, the CpG and saponin may be in free suspension or may be associated via a carrier, for example a particulate carrier such as aluminium hydroxide or by a cationic liposome or ISCOM.

An example of an adjuvant combination according to the present invention is composed of one or more CpG oligonucleotides containing at least 3, or at least 6 nucleotides between two adjacent CG motifs, together with QS21 and a particulate carrier selected from the group comprising an oil-in-water emulsion or DQ. The lipopolysacchharide may be a di or monophosphoryl lipid derivative. The lipopolysaccharide may be 3 de-O acylated, in particular 3 de O acylated monophosphoryl Lipid A. In one embodiment, the adjuvant combination comprises CpG 2006 (SEQ ID NO: 4), or CpG 1758 (SEQ ID NO: 2) or CpG 1826 (SEQ ID NO: 1) mixed with QS21, and a particulate carrier selected from the group comprising an oil-in-water emulsion or DQ. Accordingly, vaccines of the invention may, for example, comprise such adjuvant combinations and an antigen. The vaccine of the present invention may be used to generate systemic immune responses after administration to an individual through the systemic route.

Exemplary adjuvant compositions that may form part of vaccines of the present invention are described in WO00/62800.

The adjuvant combinations of the present invention can comprise an oil based emulsion. Oil emulsion adjuvants have been known for many years, including work on Freunds complete and incomplete mineral oil emulsion adjuvants. Since that time much work has been performed to design stable and well tolerated alternatives to these potent, but reactogenic, adjuvant formulations.

Many single or multiphase emulsion systems have been described. Oil in water emulsion adjuvants per se have been suggested to be useful as adjuvant compositions (EP 0 399 843B), also combinations of oil in water emulsions and other active agents have been described as adjuvants for vaccines (WO 95/17210; WO 98/56414; WO 99/12565; WO 99/11241). Other oil emulsion adjuvants have been described, such as water in oil emulsions (U.S. Pat. No. 5,422,109; EP 0 480 982 B2) and water in oil in water emulsions (U.S. Pat. No. 5,424,067; EP 0 480 981 B).

The oil emulsion adjuvants for use in the present invention may be natural or synthetic, and may be mineral or organic. Examples of mineral and organic oils will be readily apparent to the man skilled in the art.

In order for any oil in water composition to be suitable for human administration, the oil phase of the emulsion system may comprise a metabolisable oil. The meaning of the term metabolisable oil is well known in the art. Metabolisable can be defined as “being capable of being transformed by metabolism” (Dorland's Illustrated Medical Dictionary, W.B. Sanders Company, 25th edition (1974)). The oil may be any vegetable oil, fish oil, animal oil or synthetic oil, which is not toxic to the recipient and is capable of being transformed by metabolism. Nuts (such as peanut oil), seeds, and grains are common sources of vegetable oils. Synthetic oils are also part of this invention and can include commercially available oils such as NEOBEE® and others. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is an oil suitable for use in this invention. Squalene is a metabolisable oil virtue of the fact that it is an intermediate in the biosynthesis of cholesterol (Merck index, 10th Edition, entry no. 8619).

Examples of oil emulsions for use in the present invention are oil in water emulsions, and in particular squalene in water emulsions.

In addition, oil emulsion adjuvants of the present invention may comprise an antioxidant, which may be the oil α-tocopherol (vitamin E, EP 0 382 271 B1).

WO 95/17210 and WO 99/11241 disclose emulsion adjuvants based on squalene, α-tocopherol, and TWEEN 80, optionally formulated with the immunostimulants QS21 and/or 3D-MPL. WO 99/12565 discloses an improvement to these squalene emulsions with the addition of a sterol into the oil phase. Additionally, a triglyceride, such as tricaprylin (C27H50O6), may be added to the oil phase in order to stabilise the emulsion (WO 98/56414).

The size of the oil droplets found within the stable oil in water emulsion may be less than 1 micron, may be in the range of substantially 30-600 nm, for example substantially around 30-500 nm in diameter, and for example substantially 150-500 nm in diameter, and in particular about 150 nm in diameter as measured by photon correlation spectroscopy. In this regard, 80% of the oil droplets by number should be within these exemplified ranges, or for example more than 90% or more than 95% of the oil droplets by number should be within the defined size ranges. The amounts of the components present in the oil emulsions of the present invention are conventionally in the range of from 2 to 10% oil, such as squalene; and when present, from 2 to 10% alpha tocopherol; and from 0.3 to 3% surfactant, such as polyoxyethylene sorbitan monooleate. The ratio of oil: alpha tocopherol may be equal or less than 1 as this provides a more stable emulsion. Span 85 may also be present at a level of about 1%. In some cases it may be advantageous that the vaccines of the present invention will further contain a stabiliser.

The method of producing oil in water emulsions is well known to the man skilled in the art. Commonly, the method comprises the mixing the oil phase with a surfactant such as a PBS/TWEEN80™ solution, followed by homogenisation using a homogenizer, it would be clear to a man skilled in the art that a method comprising passing the mixture twice through a syringe needle would be suitable for homogenising small volumes of liquid. Equally, the emulsification process in microfluidiser (M110S microfluidics machine, maximum of 50 passes, for a period of 2 minutes at maximum pressure input of 6 bar (output pressure of about 850 bar)) could be adapted by the man skilled in the art to produce smaller or larger volumes of emulsion. This adaptation could be achieved by routine experimentation comprising the measurement of the resultant emulsion until a preparation was achieved with oil droplets of the required diameter.

The vaccines of the present invention may be administered through the systemic or parenteral route such as intramuscular, intradermal, transdermal, subcutaneous, intraperitoneal or intravenous administration.

The systemic vaccine preparations of the present invention may be used to protect or treat a mammal susceptible to, or suffering from cancer, by means of administering said vaccine by intramuscular, intraperitoneal, intradermal, transdermal, intravenous, or subcutaneous administration. The vaccines of the present invention may be used to treat individuals suffering from non-small cell lung carcinoma (NSCLC), Melanoma, or Bladder cancer.

Accordingly there is provided a method for inducing an immune response against MAGE-3 and NY-ESO-1 in an individual, comprising the administration of a vaccine according to the present invention to the individual.

The amount of saponin for use in the adjuvants of the present invention may be in the region of 1-1000 μg per dose, for example 1-500 μg per dose, or for example 1-250 μg per dose, or for example between 1 to 100 μg per dose. The ratio of CpG:saponin (w/w) will, therefore, be in the range of 1:1000 to 1000:1, and will typically be in the range of 1:100 to 100:1, or for example in the range of 1:10 to 1:1 or 1:1 to 10:1. one embodiment, the ratio is 1:1, 4:1 or 10:1.

The amount of CpG or immunostimulatory oligonucleotides in the adjuvants or vaccines of the present invention is generally small, but depending on the vaccine formulation may be in the region of 1-1000 μg per dose, for example 1-500 μg per dose, or for example between 1 to 100 μg per dose.

Vaccine preparation is generally described in New Trends and Developments in Vaccines, edited by Voller et al., University Park Press, Baltimore, Md., U.S.A. 1978.

The invention therefore provides a method to prevent an individual from contracting a disease selected from the group comprising NSCLC, melanoma and bladder cancers; comprising the administration of a composition as substantially described herein through the systemic route of said individual.

Examples of suitable pharmaceutically acceptable excipients for use in the combinations of the present invention include water, phosphate buffered saline, isotonic buffer solutions.

Optionally, the vaccine adjuvant component may further comprise a derivative of LPS, such as 3D-MPL. Examples of such adjuvants include: combinations of CpG, 3D-MPL and QS21 (EP 0 671 948 B1), oil in water emulsions comprising CpG, 3D-MPL and QS21 (WO 95/17210, WO 98/56414), or 3D-MPL formulated with other carriers (EP 0 689 454 B1) in combination with the CpG oligonucleotides as herein described.

The adjuvant combinations of the present invention may include at least one enterobacterial lipopolysaccharide derived adjuvant.

It has long been known that enterobacterial lipopolysaccharide (LPS) is a potent stimulator of the immune system, although its use in adjuvants has been curtailed by its toxic effects. A non-toxic derivative of LPS, monophosphoryl lipid A (MPL), produced by removal of the core carbohydrate group and the phosphate from the reducing-end glucosamine, has been described by Ribi et al (1986, Immunology and Immunopharmacology of bacterial endotoxins, Plenum Publ. Corp., NY, p 407-419) and has the following structure:

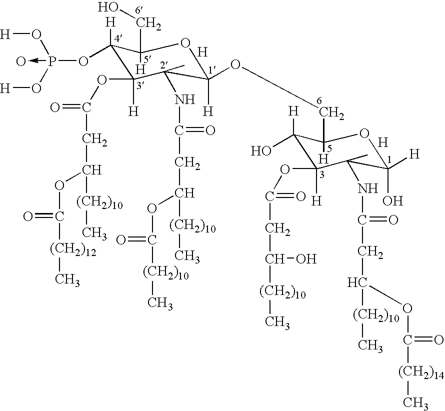

A further detoxified version of MPL results from the removal of the acyl chain from the 3-position of the disaccharide backbone, and is called 3-O-Deacylated monophosphoryl lipid A (3D-MPL). It can be purified and prepared by the methods taught in GB 2122204B, which reference also discloses the preparation of diphosphoryl lipid A, and 3-O-deacylated variants thereof. One example of a form of 3D-MPL is in the form of an emulsion having a small particle size less than 0.2 μm in diameter, and its method of manufacture is disclosed in WO 94/21292. Aqueous formulations comprising monophosphoryl lipid A and a surfactant have been described in WO 98/43670A2.

The bacterial lipopolysaccharide derived adjuvants which may be formulated in the adjuvant combinations of the present invention may be purified and processed from bacterial sources, or alternatively they may be synthetic. For example, purified monophosphoryl lipid A is described in Ribi et al 1986 (supra), and 3-O-Deacylated monophosphoryl or diphosphoryl lipid A derived from Salmonella sp. is described in GB 2220211 and U.S. Pat. No. 4,912,094. Other purified and synthetic lipopolysaccharides have been described (WO 98/01139; U.S. Pat. No. 6,005,099 and EP 0 729 473 B1; Hilgers et al., 1986, Int. Arch. Allergy. Immunol., 79(4):392-6; Hilgers et al., 1987, Immunology, 60(1):141-6; and EP 0 549 074 B1). The bacterial lipopolysaccharide adjuvants may be 3D-MPL and the β(1-6) glucosamine disaccharides described in U.S. Pat. No. 6,005,099 and EP 0 729 473 B1.

Accordingly, LPS derivatives that may be used in the present invention are those immunostimulants that are similar in structure to that of LPS or MPL or 3D-MPL. In another aspect of the present invention LPS derivatives may be an acylated monosaccharide, which is a sub-portion to the above structure of MPL.

An example of a disaccharide adjuvant is a purified or synthetic lipid A of the following formula: 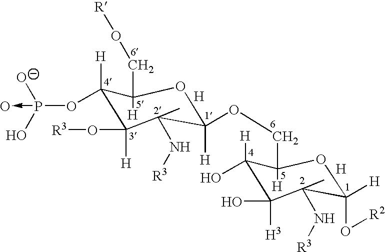
wherein R2 may be H or PO3H2; R3 may be an acyl chain or β-hydroxymyristoyl or a 3-acyloxyacyl residue having the formula:

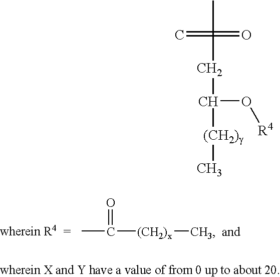

One exemplary vaccine formulation comprises a 0.5 ml adjuvant composition comprising an oil in water emulsion comprising and oil phase: about 12 mg alpha tocopherol, about 11 mg squalene, and about 5 mg tween 80; and in the aqueous phase: 50 μg 3D-MPL and 50 μg QS21 and 500 μg CpG. Another exemplary vaccine formulation comprises an oil in water emulsion comprising and oil phase: about 2 mg alpha tocopherol, about 2 mg squalene, and about 1 mg tween 80; and in the aqueous phase: 50 μg 3D-MPL and 50 μg QS21.

In another embodiment there is provided a vaccine composition comprising modified MAGE protein, as described in WO9940188; an “immunogenic region” of NY-ESO1 gene product, for example: the NY-ESO1 protein; a protein, polypeptide or peptide consisting of or comprising the C terminal portion of the protein containing the Class I and/or Class II epitopes of NY-ESO1; overlapping long peptides comprising this region; and/or, specific CD8 peptides; an immunostimulatory adjuvant comprising one or more of an alum salt, an oil-in-water emulsion (O/W emulsion).

What is claimed is:

1. A vaccine composition comprising (a) an antigen component comprising a modified MAGE-3 antigen and an NY-ESO-1 antigen, and (b) an adjuvant component comprising an immunostimulatory adjuvant selected from the group consisting of alum salt; cholesterol; oil-in-water emulsion (O/W emulsion); oil-in-water emulsion low dose; an immunostimulatory oligonucleotide; tocopherol; liposome; a saponin; and a lipopolysaccharide.

2. A vaccine as claimed in claim 1, wherein the adjuvant component comprises a combination of an immunostimulatory oligonucleotide and a saponin.

3. A vaccine as claimed in claim 1, wherein the modified MAGE-3 antigen has SEQ ID NO 1.

4. A vaccine as claimed in claim 1, wherein the NY-ESO-1 antigen has SEQ ID NO 2.

5. A vaccine as claimed in claim 1, wherein the saponin is QS21 formulated in a cholesterol containing liposome.

6. A vaccine as claimed in claim 1, wherein the immunostimulatory oligonucleotide is CpG.

7. A vaccine composition as claimed in claim 1 further comprising 3 de-o-acylated monophosphoryl lipid A.


SEQUENCE LISTING (8 sequences)

SEQ ID NO: 1 - protein, 451 aa, Artificial [sequence omitted]

SEQ ID NO: 2 - protein, 180 aa, Artificial
  NY-ESO-1 Polypeptide
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 3 - DNA, 752 bp, Artificial
  NY-ESO-1 polynucleotide sequence
atcctcgtgggccctgaccttctctctgagagccgggcagaggctccggagccatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaagcccagcctggcgccccttcctaggtcatgcctcctcccctagggaatggtcccagcacgagtggccagttcattgtgggggcctgattgtttgtcgctggaggaggacggcttacatgtttgtttctgtagaaaataaaactgagctacgaaaaa

SEQ ID NO: 4 - DNA, 20 bp, Artificial
  CpG 1826 oligonucleotide
tccatgacgttcctgacgtt

SEQ ID NO: 5 - DNA, 18 bp, Artificial
  CpG 1758 oligonucleotide
tctcccagcgtgcgccat

SEQ ID NO: 6 - DNA, 30 bp, Artificial
  CpG oligonucleotide
accgatgacgtcgccggtgacggcaccacg

SEQ ID NO: 7 - DNA, 24 bp, Artificial
  CpG 2006 oligonucleotide
tcgtcgttttgtcgttttgtcgtt

SEQ ID NO: 8 - DNA, 20 bp, Artificial
  CpG 1668 oligonucleotide
tccatgacgttcctgatgct